(19) United States
(12) Patent Application Publication
(10) Pub. No.: US 2023/0085603 A1
(43) Pub. Date: Mar. 16, 2023

(54) RED RICE FERMENT, PREPARATION METHOD THEREOF AND METHOD FOR IMPROVING SKIN CONDITION WITH THE SAME

(71) Applicant: TCI CO., LTD., Taipei (TW)

(72) Inventors: YUNG-HSIANG LIN, Taipei (TW); MARGARET LAI, Taipei (TW)

(21) Appl. No.: 17/823,520

(22) Filed: Aug. 31, 2022

Related U.S. Application Data
(60) Provisional application No. 63/238,782, filed on Aug. 31, 2021.

(51) Int. Cl. A61K 36/899; A61K 8/9794; A61Q 19/08; A61Q 19/00; A61P 17/02

(52) U.S. Cl. CPC A61K 36/899; A61K 8/9794; A61Q 19/08; A61Q 19/00; A61P 17/02; A61K 2236/19

(57) ABSTRACT

A preparation method of a red rice ferment, including obtaining a red rice ferment by fermenting a red rice extract by yeast, Lactobacillus, and Acetobacter aceti. A red rice ferment is obtained from the preparation method of a red rice ferment. A method for improving skin condition of a subject in need thereof with the red rice ferment, comprising administering to the subject an effective dose of a composition containing the red rice ferment.

### CROSS-REFERENCE TO RELATED APPLICATION

This application claims the benefit of U.S. provisional application Ser. No. 63/238,782, filed on Aug. 31, 2021. The entirety of the above-mentioned patent application is hereby incorporated by reference herein and made a part of the specification.

### BACKGROUND

### Technical Field

The present disclosure relates to application of red rice, in particular to a red rice ferment, a preparation method thereof and a method for improving skin condition with the red rice ferment.

### Related Art

Since the rise of the concept of organic and natural diets, biotechnology companies and food companies have been actively investing in the research and development of natural plant-related products. In order to scientifically prove the health benefits of plant-related products to health, active ingredient analysis and efficacy evaluation of plants have become key items in product development.

Red rice, an endemic species from Hualien, Taiwan, has been cultivated for more than 300 years, and its characteristic of being rich in anthocyanin-equivalent ingredients has earned it the reputation of confinement rice. Therefore, red rice has also become one of the objects of active research and development by biotechnology companies and food companies.

In view of this, how to extract active ingredients from red rice and what effect the red rice has are urgent issues to be solved by all parties.

### SUMMARY

In some embodiments, provided is a preparation method of a red rice ferment, including: fermenting a red rice extract by yeast, Lactobacillus and Acetobacter aced to obtain the red rice ferment.

In some embodiments, provided is a red rice ferment, obtained by the aforementioned preparation method of a red rice ferment.

In some embodiments, provided is use of a red rice ferment for preparation of a skin care composition. The red rice ferment is obtained by the preparation method of a red rice ferment.

In some embodiments, provided is a method for improving skin condition of a subject in need thereof with the red rice ferment, including administering to the subject an effective amount of a composition containing the red rice ferment. The red rice ferment is obtained by the aforementioned preparation method of a red rice ferment.

Based on the above, the preparation method of a red rice ferment according to any embodiment is suitable for preparing the red rice ferment with a skin care effect. In some embodiments, the total flavonoid content in the red rice ferment obtained after fermentation is greatly increased as compared with that before fermentation.

### BRIEF DESCRIPTION OF THE DRAWINGS

FIG. 1 is a flowchart of a preparation method of a red rice ferment;

FIG. 2 is a bar chart of total flavonoid content;

FIG. 3 is a bar chart of relative cell proliferation rate;

FIG. 4A shows photographs of appearance of wounds;

FIG. 4B is a bar chart of relative wound healing rate obtained by quantifying the results of FIG. 4A;

FIG. 5 is a bar chart of relative mitochondrial activity;

FIG. 6A shows photographs of skin texture detection results of one of subjects in a human experiment;

FIG. 6B is a bar chart of relative skin textures obtained by performing skin texture detection on 10 subjects before dosing and after dosing with the red rice ferment for 4 consecutive weeks;

FIG. 7A shows photographs of skin wrinkle detection results and naked eye observation results of one of the subjects in the human experiment;

FIG. 7B is a bar chart of relative skin wrinkles obtained by performing skin wrinkle detection on 10 subjects before dosing and after dosing with the red rice ferment for 4 consecutive weeks;

FIG. 8A shows photographs of skin pore detection results of one of the subjects in the human experiment;

FIG. 8B is a bar chart of relative skin pores obtained by performing skin pore detection on 10 subjects before dosing and after dosing with the red rice ferment for 4 consecutive weeks;

FIG. 9A shows photographs of skin redness detection results of one of the subjects in the human experiment; and

FIG. 9B is a bar chart of relative skin redness obtained by performing skin redness detection on 10 subjects before dosing and after dosing with the red rice ferment for 4 consecutive weeks.

### DETAILED DESCRIPTION

The term “red rice” used herein is scientifically called Oryza saliva.

The “sugar content” mentioned herein refers to Degrees Brix (° Bx). Degrees Brix is a unit of measurement of sugar content, which represents the number of grams of sucrose dissolved per 100 grams of aqueous solution at 20° C.

In an embodiment, provided is a preparation method of a red rice ferment, including: fermenting a red rice extract by yeast, Lactobacillus and Acetobacter aceti to obtain the red rice ferment.

In some embodiments, the red rice extract may be obtained by performing an extraction process on red rice with water containing an enzyme solution.

In some embodiments, the extraction process may be performed at a temperature of 60° C. to 100° C. for 0.5 hour to 5 hours. In an exemplary embodiment, the extraction process may be divided into two stages (hereinafter referred to as a first stage and a second stage). In the first stage, the extraction is performed at a temperature of 80° C. to 100° C. for 0.5 hour to 1 hour. In the second stage, the extraction is performed at a temperature of 60° C. to 65° C. for 1.5 hours. In another exemplary embodiment, the extraction process may be a single stage, for example, the extraction is performed at a temperature of 80° C. to 100° C. for 0.5 hour to 1 hour. In still another exemplary embodiment, the extraction process may be divided into three stages (hereinafter referred to as a first stage, a second stage and a third stage). In the first stage, the extraction is performed at a temperature of 80° C. to 100° C. for 0.5 hour to 1 hour. In the second stage, the extraction is performed at a temperature of 60° C. to 65° C. for 1.5 hours. In the third stage, the extraction is performed at a temperature of 95° C. for 2.5 hours. In some embodiments, the red rice to be extracted may be whole red rice, or broken red rice granules or milled red rice powder.

In some embodiments, a preparation method of the red rice extract is as follows. First, whole red rice is broken into red rice granules, and the red rice granules and water are mixed in a weight ratio of 1:6 to obtain a raw material mixture. Next, 1% (w/v) amylase is added to the raw material mixture (i.e., the red rice granules are soaked in the water containing 1% (w/v) amylase), and then, a sterilization process (hereinafter referred to as a first sterilization process) is performed to obtain a first extract. In some embodiments, the first sterilization process may be performed at a temperature of 80° C. to 100° C. for 0.5 hour to 1 hour. In some embodiments, the red rice may be commercially available and produced in Hualien, Taiwan. In some embodiments, the red rice granules may have a particle size of 12 millimeters (mm) or less.

In some embodiments, 1% (w/v) saccharifying enzyme solution (diastatic enzyme) may be further added to the first extract for further reaction to obtain a second extract. For instance, the first extract obtained by the sterilization process is cooled to 60° C. to 65° C., 1% (w/v) saccharifying enzyme solution is added, and then, the reaction (or common solvent extraction) is performed at 60° C. to 65° C. for 90 minutes to obtain the second extract.

In some embodiments, the second extract may be subjected to another sterilization process (hereinafter referred to as a second sterilization process) to obtain a sterilized extract. In some embodiments, the second sterilization process may be performed at a temperature of 95° C. for 2.5 hours.

Specifically, when the extraction process is divided into three stages, the first sterilization process is the aforementioned first stage, the common solvent extraction is the aforementioned second stage, and the second sterilization process is the aforementioned third stage.

In some embodiments, the amylase may be selected from α-amylase, β-amylase, γ-amylase (glucoamylase) and isoamylase, or a combination thereof. In some embodiments, the amylase may be glucoamylase (glucan 1,4-alpha-glucosidase).

In some embodiments, the red rice extract may be the aforementioned first extract, the aforementioned second extract, or the aforementioned sterilized extract.

In some embodiments, after the red rice extract is cooled to room temperature, the yeast, the Lactobacillus and the Acetobacter aceti may be directly added in different stages without filtering out solids (for example, the red rice as the raw material for extraction) in the red rice extract, and fermentation is performed for 5 days to 15.5 days to obtain the red rice ferment.

In some embodiments, the content of the aforementioned yeast is 0.5% (w/v) of the red rice extract. The content of the aforementioned Lactobacillus is 0.1% (w/v) of the red rice extract. The content of the aforementioned Acetobacter aceti is 5% (w/v) of the red rice extract.

In some embodiments, the ratio of fermentation days of the yeast to the Lactobacillus to the Acetobacter aceti may be (1 to 2.5):(1 to 3):(3 to 10), preferably 1:1:5. In some embodiments, the fermentation temperature may be 28° C. to 37° C., preferably 30° C.

In some embodiments, the yeast may be Saccharomyces cerevisiae. For example, the Saccharomyces cerevisiae is deposited in the Bioresource Collection and Research Center of the Food Industry Research and Development Institute with an accession number BCRC 20271 (also deposited in the American Type Culture Collection with an international accession number ATCC26602), or is another commercially available Saccharomyces cerevisiae strain or Saccharomyces cerevisiae isolated and purified from natural sources by using a conventional microbial isolation method in the art.

In some embodiments, the Lactobacillus may be Lactobacillus plantarum. For example, the Lactobacillus plantarum is deposited in the Bioresource Collection and Research Center of the Food Industry Research and Development Institute with an accession number BCRC 910760 (also deposited in the Deutsche Sammlung von Mikroorganismen und Zellkulturen (DSMZ) with an international accession number DSM32451), or is another commercially available Lactobacillus plantarum strain or Lactobacillus plantarum isolated and purified from natural sources by using a conventional microbial isolation method in the art.

In some embodiments, the Acetobacter aceti is deposited in the Bioresource Collection and Research Center of the Food Industry Research and Development Institute with a conventional number BCRC 11688 (also deposited in the American Type Culture Collection with an international accession number ATCC15973), or is other commercially available Acetobacter aceti strains or Acetobacter aceti isolated and purified from natural sources by using a conventional microbial isolation method in the art.

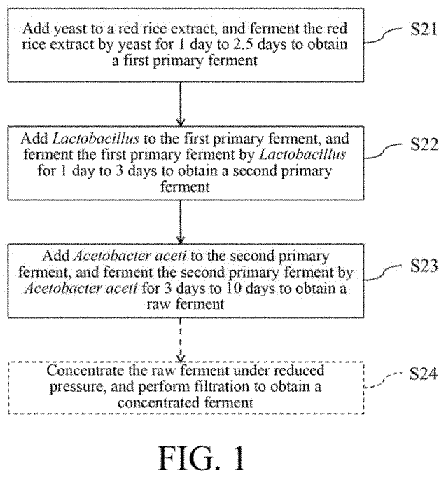

For example, referring to FIG. 1, after the red rice extract was cooled to room temperature, 0.5% (w/v) yeast was firstly added to the red rice extract, and the mixture of the red rice extract and the yeast was fermented at 28° C. to 37° C. for 1 day to 2.5 days to form a first primary ferment (step S21).

After the first primary ferment was formed, 0.1% (w/v) Lactobacillus was added to the first primary ferment, and the mixture of the first primary ferment and the Lactobacillus was fermented at 28° C. to 37° C. for 1 day to 3 days to form a second primary ferment (step S22).

After the second primary ferment was formed, 5% (w/v) Acetobacter aceti was added to the second primary ferment, and the mixture of the second primary ferment and the Acetobacter aceti was fermented at 28° C. to 37° C. for 3 days to 10 days to form a raw ferment (step S23).

In some embodiments, after the raw ferment is formed, concentration and/or filtration may be further performed.

In some embodiments, after the raw ferment was formed, the raw ferment was concentrated under reduced pressure at 55° C. to 65° C. and filtered with a filter screen with suitable mesh to obtain a concentrated ferment (step S24). In some embodiments, the suitable mesh may be 200 mesh to 400 mesh.

In some embodiments, the red rice ferment may be the aforementioned first primary ferment, the aforementioned second primary ferment, the aforementioned raw ferment, or the aforementioned concentrated ferment.

In some embodiments, the red rice ferment has a pH of less than 3.5 and greater than 0 (error ±1.0), and the red rice ferment has a sugar content of 2 to 3.5° Bx (error ±2).

In some embodiments, the red rice ferment has a skin care effect. In some embodiments, the red rice ferment can enhance the mitochondrial activity of subject's skin fibroblasts, delay skin aging, resist wrinkles and alleviate skin redness. In some embodiments, the red rice ferment can alleviate subject's skin roughness, firm skin and minimize skin pores. In some embodiments, the red rice ferment has the effects of promoting regeneration of subject's skin fibroblasts, promoting wound healing and reducing wound area. The subject includes animals, preferably humans.

In some embodiments, the red rice ferment is used for enhancing mitochondrial activity of cells. It is generally believed by the academic community that higher mitochondrial activity indicates a lower physiological age of cells. High mitochondrial activity can also assist cells in regulating the balance of electrolytes (such as sodium, potassium and calcium).

In some embodiments, the red rice ferment can be used for preparation of a composition for skin care. The skin care includes enhancing mitochondrial activity of skin fibroblasts, delaying skin aging, preventing wrinkles, alleviating skin redness, alleviating skin roughness, firming skin, minimizing skin pores promoting regeneration of skin fibroblasts, promoting wound healing, reducing wound area, or a combination thereof.

In some embodiments, any of the aforementioned compositions may be an edible composition. In other words, the edible composition includes a specific content of the red rice ferment. In some embodiments, the aforementioned edible composition may be a food product or a food additive. In some embodiments, the food product may be, but not limited to, beverages, fermented foods, bakery products, health foods or dietary supplements.

In some embodiments, the aforementioned of edible composition may be administered to the subject orally. The edible composition may be in the form of powder, granules, a solution, a colloid or a paste.

In some embodiments, any of the aforementioned compositions may be a cosmetic or a skin care product. In other words, the cosmetic or the skin care product includes a specific content of the red rice ferment.

In some embodiments, the aforementioned cosmetic or skin care product may be in any of the following forms: a toner, a gel, a jelly mask, a clay mask, a lotion, a cream, a lipstick, a foundation, a compact powder, a loose powder, a cleansing oil, a cleansing milk, a facial cleanser, a body wash, a shampoo, a hair conditioner, a sun block, a hand cream, a nail polish, a perfume, a serum and a mask.

In some embodiments, the aforementioned cosmetic or skincare product may further include an acceptable ingredient for external use products as needed. In some embodiments, the acceptable ingredient for external use products may be, for example, an emulsifier, a penetration enhancer, a softening agent, a solvent, an excipient, an antioxidant or a combination thereof.

In some embodiments, the aforementioned cosmetic or skin care product may be administered to the subject externally. The cosmetic or skin care product may be in the form of powder, granules, a solution, a colloid or a paste.

In some embodiments, the subject may be a human.

### Example 1: Preparation of Red Rice Ferment

(1-1) Preparation of Red Rice Extract

Whole red rice was broken into red rice granules having a particle size of less than 12 mm, and the red rice granules and water were mixed in a weight ratio of 1:6 to obtain a raw material mixture.

Then, 1% (w/v) amylase was added to the raw material mixture to obtain the raw material mixture containing amylase. The amylase is glucoamylase.

Next, the raw material mixture containing amylase was sterilized at 95° C. for 1 hour to obtain a primary extract containing solids.

The primary extract containing solids was cooled to 60° C., then 1% (w/v) saccharifying ferment was added, and the reaction was performed at 60° C. for 90 minutes to obtain the red rice extract.

(1-2) Preparation of Red Rice Ferment

First, 0.5% (w/v) Saccharomyces cerevisiae was added to the red rice extract, and the mixture of the red rice extract and the Saccharomyces cerevisiae was fermented at 30° C. for 1 day to obtain a first primary ferment. Moreover, the first primary ferment had a pH of less than 4 and a sugar content of about 9° Bx. The Saccharomyces cerevisiae was purchased from BCRC (with an accession number BCRC 20271). Here, the red rice extract was fermented under the action of the Saccharomyces cerevisiae to produce ethanol such that active ingredients in the red rice were extracted.

0.1% (w/v) Lactobacillus plantarum was added to the first primary ferment, the first primary ferment was fermented under the action of Lactobacillus plantarum at 30° C. for 1 day to obtain a second primary ferment. The Lactobacillus plantarum was purchased from BCRC (with an accession number BCRC910760). Moreover, the obtained second primary ferment had a pH of less than 3.5 and a sugar content of about 6° Bx. Here, the Lactobacillus was used to further consume glucose in the first primary ferment to reduce the sugar content of the solution and produce lactic acid, such that the pH of the first primary ferment was reduced, thereby facilitating further extraction of other different active ingredients in the red rice.

5% (w/v) Acetobacter aceti was added to the second primary ferment, and the second primary ferment was fermented under the action of the Acetobacter aceti at 30° C. for 5 days to obtain a raw ferment. The Acetobacter aceti was purchased from BCRC (with an accession BCRC11688). Moreover, the obtained raw ferment had a pH of less than 3.5 and a sugar content of about 3.5° Bx. Here, the Acetobacter aceti was used to consume ethanol in the second primary ferment to further reduce the glucose content.

The raw ferment was concentrated under reduced pressure at 60° C., and then filtered through a 200-mesh filter screen to obtain a concentrated ferment. Moreover, the obtained concentrated ferment had a pH of less than 3.5 and a sugar content of about 2° Bx.

Finally, an oligosaccharide solution containing 30% (w/v) isomaltooligosaccharide was added to the concentrated ferment until the sugar content of the concentrated ferment reached 24° Bx, thereby obtaining the red rice ferment used for subsequent experiments.

### Example 2: Total Flavonoid Content of Red Rice Ferment

(2-1) Experimental Materials

(a) 10% (v/v) Aluminum nitrate (Alfa Aesar 12360) (aqueous solution)

(b) 5% (v/v) Sodium nitrite (Sigma 31443) (aqueous solution)

(c) 4% (v/v) Sodium hydroxide (NaOH, Macron 7708-10) (aqueous solution)

(d) 200 μg/mL Rutin standard solution (methanol solution)

(2-2) Experimental Procedure for Creating Standard Curve

0 μL, 200 μL, 400 μL, 600 μL, 800 μL, 1000 μL and 1200 μL of the above rutin standard solution were respectively uniformly mixed with 1200 μL, 1000 μL, 800 μL, 600 μL, 400 μL, 200 μL and 0 μL of water to form 7 rutin solutions with different concentrations respectively.

200 μL of rutin solutions with different concentrations were added to their respective test tubes. 200 μL of 5% (v/v) sodium nitrite was added to each rutin solution, and the mixture was uniformly mixed and allowed to stand for 6 minutes.

Then, 200 μL of 10% (v/v) aluminum nitrate was added to each test tube, and then the mixture was uniformly mixed and allowed to stand for 6 minutes.

2 mL of 4% (v/v) sodium hydroxide was added to each test tube, and the mixture was uniformly mixed. Then 1.4 mL of H₂O was added, and the mixture was uniformly mixed to form a reaction solution (in each test tube).

Next, 200 μL of reaction solution was taken from each test tube and added to a 96-well plate, then the absorbance was measured at O.D.500 nm using a spectrophotometer, and a standard curve was drawn.

(2-3) Experimental Procedure for Testing Samples

The red rice extract obtained in Example 1 was used as a sample of a positive control group (also called a contrast group), and the red rice ferment obtained in Example 1 was used as a sample of the experiment group.

After each sample was diluted properly, 200 μL of the diluted sample was taken and added to a test tube. Here, each sample was tested at least in triplicates.

200 μL of 5% (v/v) sodium nitrite was added to each test tube, and the mixture was uniformly mixed and allowed to stand for a first duration of 6 minutes.

After the standing for the first duration, 200 μL of 10% (v/v) aluminum nitrate was added to each test tube, and the mixture was uniformly mixed and allowed to stand for a second time for 6 minutes.

After the standing for the second duration, 2 mL of 4% (v/v) sodium hydroxide was added to each test tube, the mixture was uniformly mixed, then 1.4 mL of water was added for uniformly mixing to form a reaction solution (in each test tube).

200 μL of the above reaction solution was taken from each test tube and added to a 96-well plate, and the absorbance was measured at O.D.500 nm using a spectrophotometer.

Based on the standard curve obtained in (2-2) and the absorbance of the sample measured, the concentration of the sample was calculated using an interpolation method, thereby obtaining the original total flavonoid content of each sample.

(2-4) Experimental Results

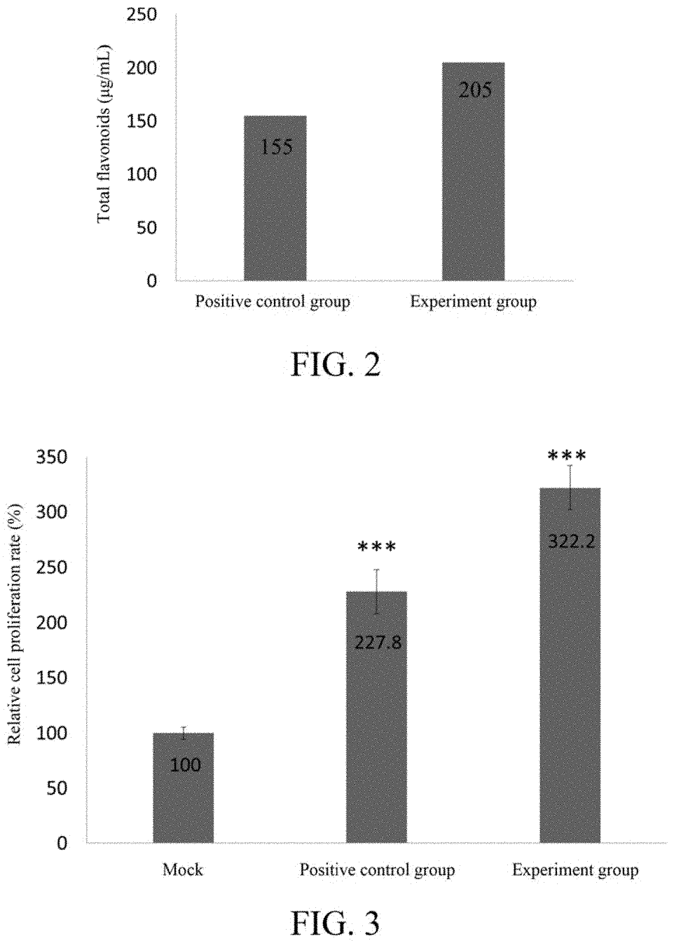

Referring to FIG. 2, the total flavonoid content measured in the positive control group (i.e., the red rice extract) was 155 μg/mL, and the total flavonoid content measured in the experiment group (i.e., the red rice ferment) was 205 μg/mL. In other words, as compared with the positive control group, the total flavonoid content in the fermentation-treated experiment group was significantly increased (by 1.3 folds).

The results of this experiment showed that the red rice ferment obtained after the fermentation released high levels of total flavonoids as compared with that before fermentation. The flavonoids had the abilities of scavenging free radicals and antioxidation. In other words, the experiment showed that as compared with the red rice extract, the total flavonoid content of the red rice ferment was greatly increased. Therefore, the red rice ferment had the effects of scavenging free radicals, antioxidation and antiaging.

### Example 3: Ability of Red Rice Ferment to Promote Skin Cell Proliferation

(3-1) Materials and Instruments

(a) Cell line: human skin fibroblasts (CCD-996SK, type: human skin fibroblast) (BCRC: 60153).

(b) Cell culture medium: minimum essential medium (MEM) (Gibco; Cat. No. 11095080) containing 10% (v/v) Fetal Bovine Serum (FBS) (Gibco; Cat. No. 10437-028), 1% (v/v) penicillin-streptomycin (Gibco; Cat. No. 15140122) and 1 mM sodium pyruvate (Gibco; Cat. No. 11360-070).

(c) Cell proliferation enzyme-linked immunosorbent assay (ELISA) kit (purchased from Roche; Cat. No. 11647229001).

(d) Test samples: the red rice extract obtained in Example 1 and the red rice ferment obtained in Example 1.

(3-2) Experimental Procedure

First, human fibroblasts were inoculated in a 96-well cell culture plate, 3×10³ cells/well, and cultured at 37° C. for 2 hours. Here, the experiment was performed in three groups (as shown in Table I), and each group was tested in triplicates.

Next, 100 μL of experimental medium was added to each well corresponding to its group (as shown in Table I below), and 10 μL of 100 μM bromodeoxyuridine (BrdU) was added to each well. Then the cells were cultured at 37° C. for 24 hours, and the cell proliferation was detected based on the content of BrdU incorporated into DNA.

TABLE I

| Group | Experimental medium |
|---|---|
| Mock | Cell culture medium without any test samples |
| Positive | Cell culture medium containing 0.125% (v/v) red rice |
| control group | extract obtained in Example 1 |
| Experiment | Cell culture medium containing 0.125% (v/v) red rice |
| group | ferment obtained in Example 1 |

The experimental medium in each well was removed without disturbing adherent cells, and 200 μL of fixative (FixDenat) was added to each well, followed by incubation at room temperature for 30 minutes.

Next, the fixative in each well was removed, and the cells were rinsed once with phosphate buffered saline (PBS (1×)).

After the completion of the rinsing, 100 μL of Anti-BrdU-POD (Anti-BrdU POD stock solution: Antibody dilution solution=1:100) was added to each well, followed by the incubation at room temperature for 90 minutes. Here, peroxidase-labeled Anti-BrdU was used to identify the BrdU.

After the incubation, the residual peroxidase-labeled Anti-BrdU was removed, and the cells were rinsed with 200-300 μL of cleaning solution for 3 times.

After the completion of the rinsing, 100 μL of matrix solution (tetramethylbenzidine, TMB) was added to each hole, and allowed to stand at room temperature for about 5 to 30 minutes until the reaction solution became colored.

Finally, 25 μL of 1 M sulfuric acid was added, and the plate was shaken at 300 rpm for about 1 minute to stop the reaction.

After the reaction was stopped, an ELISA reader (BioTek, USA) was used to measure the absorbance of the sample at O.D.450 nm.

For the aforementioned experimental procedure, reference can be made to the Instruction Manual of the cell proliferation ELISA kit.

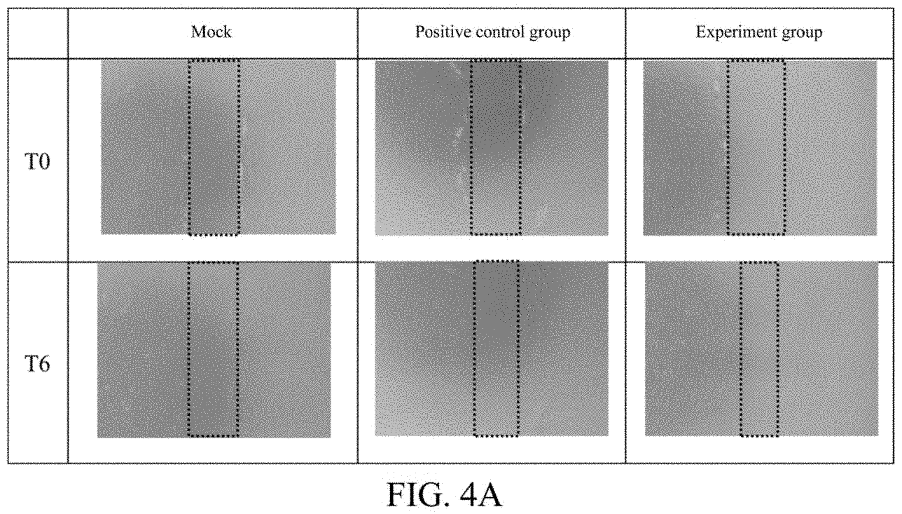

Here, the experimental results were expressed as mean±standard deviation (SD), and differences between the groups were statistically analyzed by student's t-test. In FIG. 3, *** represents a large significant difference (p<0.001) as compared with the mock.

(3-3) Experimental Results

Referring to FIG. 3, the cells in the mock were cultured in a medium without any test samples, that is, the human fibroblasts in the mock were under normal physiological metabolism. Here, the cell proliferation rate of the mock was set as 100%. The cells in the positive control group were cultured in a medium containing the red rice extract. Relative to the mock, the skin cell proliferation rate observed in the positive control group was 227.8%. The cells in the experiment group were cultured in a medium containing the red rice ferment. Relative to the mock, the skin cell proliferation rate observed in the Experiment group was 322.2%.

As can be seen, as compared with the positive control group, the relative skin cell proliferation rate observed in the experiment group was significantly increased (by 1.4 folds). Therefore, the red rice ferment obtained after fermentation had the effect of greatly promoting skin cell proliferation as compared with that before fermentation. The red rice ferment had the ability of accelerating skin tissue regeneration to strengthen skin barrier.

### Example 4: Wound Healing Assay

(4-1) Materials and Instruments

(a) Cell line: human skin fibroblasts (CCD-996SK, type: human skin fibroblast) (BCRC: 60153).

(b) Cell culture medium: minimum essential medium (Eagle) (MEM (Eagle)) (Gibco, Cat. No. 11095080) with 10% (v/v) fetal bovine serum (Gibco; Cat. No. 10437-028). The minimum essential medium (Eagle) was prepared from Eagle's balance slat solution (Eagle's BSS) with additional components to make it contain 1 mM sodium pyruvate, 1.5 g/L sodium bicarbonate and 0.1 mM non-essential amino acid solution.

Experimental Procedure

(a) First, thin film membrane cell culture dishes (also referred to as culture-Insert wells (SPL®SPLInsert™) were placed in a 24-well plate (GeneDireX), and then human skin fibroblasts were inoculated into the wells, 1.5×10⁵ cells/well, and cultured overnight at 37° C. after addition of the cell culture medium.

(b) After the thin film membrane cell culture dishes were removed from each well of the 24-well plate, a cell gap having roughly the same size was observed on the cell monolayer in each well. Here, the formed cell gap was used to simulate a wound.

(c) The cell culture medium in each well was removed, and the cells were rinsed with PBS (1×) (Gibco) once.

(d) The rinsed human skin fibroblasts were divided into three groups (as shown in Table II) (triplicates per group), followed by the addition of the experimental medium (as shown in Table II). Then, cell images were obtained using a microscope (ZEISS) and a camera, the migration distance of the human skin fibroblasts (i.e., the gap area at 0 h (T0)) were observed and recorded, and then the cells were cultured at 37° C. for additional 6 hours. Moreover, the gap area at 0 h (T0) was taken as the initial wound area (i.e., as the base level).

(e) After 6 hours of culture, cell images were obtained also using the microscope and the camera, and the migration distance of the human skin fibroblasts (i.e., the gap area at 6 h (T6)) were observed and recorded. Here, the gap area at 6 h (T6) was taken as the wound area after 6 hours of repair.

(f) Here, the area of the cell gap (hereinafter referred to as the wound area) in the cell image was measured using software Image J. The wound healing rate (%) was calculated based on the wound area according to the following Formula 1 to represent the distance of migration of human skin fibroblasts to the cell gap.

Here, the experimental data were expressed as mean±standard deviation (SD), and differences between the groups were statistically analyzed by student's t-test.

TABLE II

| Group | Experimental medium |
|---|---|
| Mock | Minimum essential medium (Eagle) without any test |
| | samples |
| Positive | Minimum essential medium (Eagle) containing 0.125% |
| control group | (v/v) red rice extract obtained in Example 1 |
| Experiment | Minimum essential medium (Eagle) containing 0.125% |
| group | (v/v) red rice ferment obtained in Example 1 |

The calculation formula of the wound healing rate (%) is:

$$Would healing rate ( \% ) = \frac{( wound area at time point T 0 - wound area at time point T 6 )}{( wound area at time point T 0 )} \times 100 \qquad Formula 1$$

The wound area at time point T0 was the gap area at 0 h. The wound area at time point T6 was the gap area after 6 hours of culture. Taking the gap area at time point T0 as the initial wound area, after a period of culture, the wound area gradually decreased. The wound healing rate (%) was quantified in this way: the wound area after a period of culture (i.e., the wound area at time point T6) was subtracted from the wound area at time point T0 to obtain an area difference, and this area difference was divided by the wound area at time point T0, which was expressed as a percentage. This area difference was the area of migration of human skin fibroblasts to the gap (area of wound healing).

(4-2) Experimental Results

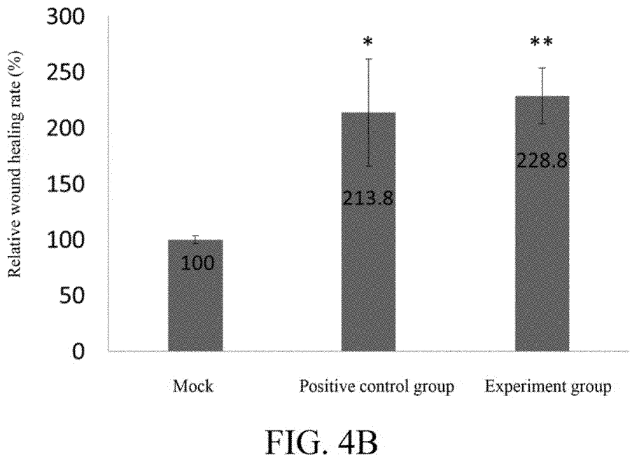

Referring to FIG. 4A, FIG. 4A showed photos of appearance of a wound in the wound healing assay, which showed the area of migration of human skin fibroblasts to the gap in each group measured using the software Image J at time point T0 and time point T6. It was noted that at time point T0 in FIG. 4A, the area surrounded by the black dotted line was the original gap area in each group (regarded as the initial wound area), and on the two sides outside the area surrounded by the black dotted line were the human fibroblasts; and at time point T6 in FIG. 4A, the area surrounded by the black dotted line was the gap area at time point T6 in each group (regarded as the wound area at time point T6). As can be seen from FIG. 4A, in the mock, from the appearance of the wound, the wound area at time point T6 was not significantly reduced as compared with the wound area at time point T0; in the positive control group, from the appearance of the wound, the wound area at time point T6 was reduced as compared with the wound area at time point T0; and in the experiment group, from the appearance of the wound, the wound area at time point T6 was significantly reduced as compared with the wound area at time point T0.

FIG. 4B shows relative wound healing rate obtained by quantifying the results of FIG. 4A. In FIG. 4B, * represents a statistically significant difference (p<0.05) as compared with the mock, and ** represents a statistically large significant difference (p<0.01) as compared with the mock.

Referring to FIG. 4B, the cells in the mock were cultured in a medium without any test samples, that is, the human fibroblasts in the mock were under normal physiological metabolism. Here, the wound healing rate of the mock was set as 100%. The cells in the positive control group were cultured in a medium containing the red rice extract. Relative to the mock, the wound healing rate observed in the positive control group was 213.8%. The cells in the experiment group were cultured in a medium containing the red rice ferment. Relative to the mock, the wound healing rate observed in the experiment group was 228.8%.

As can be seen, in the case of the same culture time, as compared with the mock, the relative wound healing rate of human fibroblasts observed in the experiment group was significantly increased (by 2.3 folds). Similarly, in the case of the same culture time, as compared with the positive control group, the relative wound healing rate of human fibroblasts observed in the experiment group was significantly increased (by 1.1 folds). Therefore, the red rice ferment obtained after fermentation had the ability of greatly increasing the area of migration of human fibroblasts to the gap and had the effect of promoting repair of human fibroblasts as compared with that before fermentation.

### Example 5: Ability of Red Rice Ferment to Enhance Mitochondrial Activity of Skin Cells

(5-1) Materials

(a) Cell line: human skin fibroblasts (CCD-996SK, type: human skin fibroblast) (BCRC: 60153).

(b) Cell culture medium: minimum essential medium (MEM) (Gibco; Cat. No. 11095080) containing 10% (v/v) fetal bovine serum (Gibco; Cat. No. 10437-028), 1% (v/v) penicillin-streptomycin (Gibco; Cat. No. 15140122), 1 mM sodium pyruvate (Gibco; Cat. No. 11360-070), 1.5 g/L sodium bicarbonate (Sigma; Cat. No. S5761-500G) and 0.1 mM non-essential amino acid solution (Gibco; Cat. No. 11140050).

(c) Test samples: the red rice extract obtained in Example 1 and the red rice ferment obtained in Example 1.

(5-2) Experimental Procedure

(a) First, human fibroblasts were inoculated into a 6-well cell culture plate at 1×10⁵ cells/well (triplicates per group), and each well contained 2 mL of cell culture medium.

(b) After the cells were cultured at 37° C. for 24 hours, the cells were divided into 3 groups (as shown in Table III), and the cell culture medium in each group was changed to the experimental medium (as shown in Table III). Cells in each group were successively cultured at 37° C. for another 24 hours.

(c) After the culture was completed, each group was subjected to mitochondrial activity analysis according to the instruction manual attached to the mitochondrial membrane potential detection kit. Mitochondrial membrane potential was measured using the mitochondrial membrane potential detection kit (BD™ MitoScreen (JC-1) kit, Cat. No. 551302), and relative fluorescence signals of aggregates of fluorescent dye JC-1 indicating mitochondrial membrane potential were detected using an Accuri C6 Plus flow cytometry (BD™, USA) (the wavelength of excitation light was about 488 nm, and the detection wavelength of scattered light was about 590 nm). The more the fluorescent signals of JC-1 aggregates indicating mitochondrial membrane potential were detected, the greater the potential difference of the inner mitochondrial membrane, the higher the mitochondrial activity was considered. The experimental data were expressed as mean±standard deviation (SD), and differences between the groups were statistically analyzed by student's t-test.

TABLE III

| Group | Experimental medium |
|---|---|
| Mock | Cell culture medium without any test samples. |
| Positive | Cell culture medium containing 0.125% (v/v) red rice |
| control group | extract obtained in Example 1. |
| Experiment | Cell culture medium containing 0.125% (v/v) red rice |
| group | ferment obtained in Example 1. |

(5-3) Experimental Results

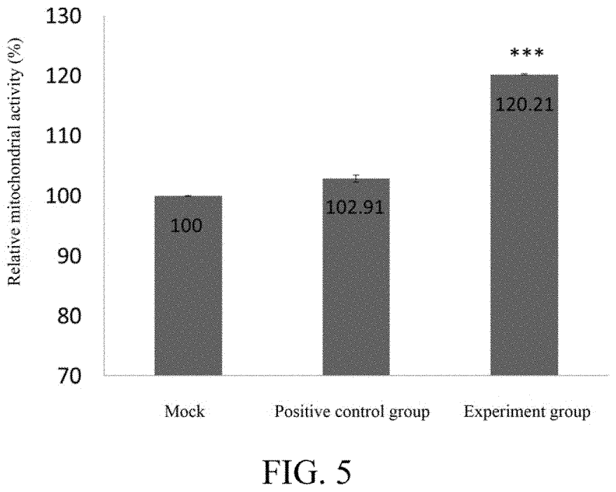

Referring to FIG. 5, in the mock, the cells were cultured in a medium without any test samples, that is, the human fibroblasts in the mock were under normal physiological metabolism. The mitochondrial activity of the mock was set as 100%. The cells in the positive control group were cultured in a medium containing the red rice extract. Relative to the mock, the mitochondrial activity observed in the positive control group was 102.91%. The cells in the experiment group were cultured in a medium containing the red rice ferment. Relative to the mock, the mitochondrial activity observed in the experiment group was 120.21%. In FIG. 5, *** represents a large significant difference (p<0.001) as compared with the mock.

As can be seen, in the case of the same culture time, as compared with the positive control group, the mitochondrial activity observed in the experiment group was significantly increased (by 1.2 folds). The experimental result showed that the red rice ferment having the antioxidation activity could enhance the mitochondrial activity of skin cells, so that the skin cells had enough energy to perform specific physiological functions or proliferate to replace old cells. Mitochondria are involved in redox and biochemical metabolic reactions of cells, and serve as an important organelle that supplies energy to cells. However, endogenous free radicals are generated when the mitochondria in cells undergo respiratory electron transfer. Damage of exogenous and endogenous free radicals to the mitochondria is considered to be the main cause of aging. As can be seen from the experiment, the red rice ferment could enhance mitochondrial activity of subject's skin cells, and thus had the antiaging effect of improving skin cells.

### Example 6: Detection of Numerical Values of Human Skin

(6-1) Experiment Preparation

(a) Test dose: 6.5 mL of red rice ferment obtained in Example 1/day.

(b) Number subjects and conditions: 10 subjects, aged from 20 to 55, with loose skin, large skin pores and/or skin redness.

(6-2) Experimental Procedure

The subjects were dosed with one bottle of test sample after breakfast every day for 4 consecutive weeks. The subjects were respectively subjected to detection of numerical values of facial skin before dosing (before dosing with the test sample, regarded as week 0) and after dosing (after dosing with the test sample for 4 consecutive weeks, regarded as week 4). Corresponding instruments and measurement methods were used depending on different detection items. Here, the detection items were (6-2-1) skin texture detection, (6-2-2) skin wrinkle detection, (6-2-3) skin pores detection and (6-2-4) skin redness detection.

When the detection of numerical values of facial skin before and after dosing was performed, the whole face of the subject should be cleaned first. Besides, the ambient temperature of the test area where the subject was located should be consistent with the average body temperature of the subject, and the ambient humidity of the test area where the subject was located should be suitable for human skin, so as to reduce the external influence of factors such as temperature and humidity in the test area on subject's skin.

(6-2-1) Skin Texture Detection

(a) Measurement Method

The facial skin of the subjects was detected using the VISIA high-end digital skin analyzer sold by Canfield, USA. The same facial skin of the same subject before and after the test was photographed under visible light (white light) using the high-resolution camera in the skin analyzer to obtain photos of the facial skin, and roughness analysis was performed according to pits and bumps using built-in software of the skin analyzer to obtain the corresponding quantified skin texture (or called skin roughness).

(b) Experimental Results

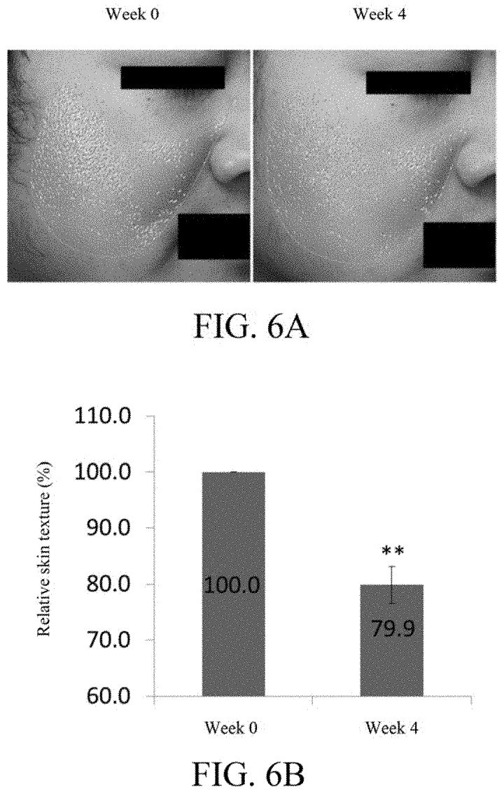

FIG. 6A showed photos of skin texture detection results of one of the subjects. As compared with week 0, the photo of facial skin at week 4 showed significantly fewer position marks of pits and bumps detected in the same area of facial skin.

Referring to FIG. 6B, the mean skin texture of the 10 subjects before dosing (week 0) was considered as 100%. After dosing with the red rice ferment every day for 4 consecutive weeks, the mean skin texture of the 10 subjects was 79.9%. That is, dosing with 6.5 mL of red rice ferment every day effectively improved the skin texture by up to 20.1%. Based on this, the red rice ferment had the effect of improving the skin texture of the subject. In FIG. 6B, ** represents a large significant difference (p<0.01) as compared with the skin texture of the subjects at week 0.

(6-2-2) Skin Wrinkle Detection

(a) Measurement Method

The facial skin of the subjects was detected using the VISIA high-end digital skin analyzer sold by Canfield, USA. The same facial skin of the same subject before and after the test was photographed under visible light (white light) using the high-resolution camera to obtain photos of the facial skin, and analysis was performed according to lengths and depths of wrinkles using built-in software of the skin analyzer to obtain the corresponding quantified skin wrinkles.

(b) Experimental Results

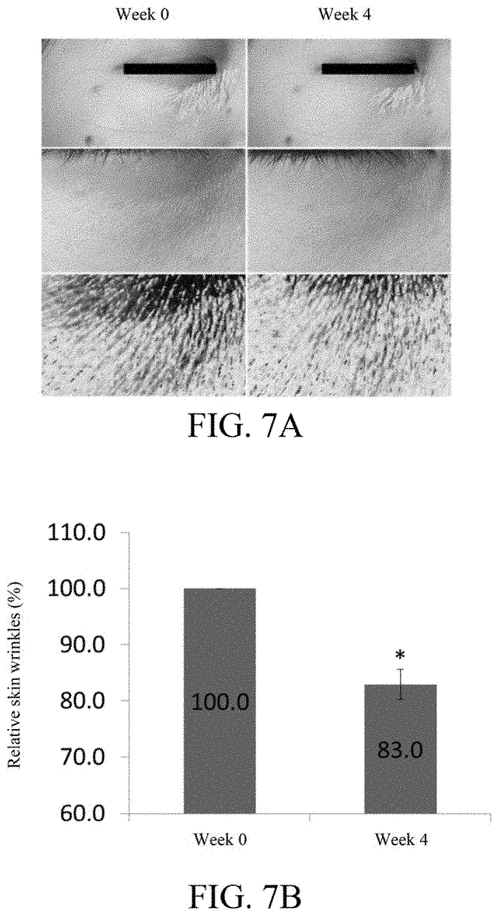

FIG. 7A showed photos of skin wrinkle detection results and naked eye observation results of one of the subjects. The photos at the top showed the detection result of the large-range observed area. The photos in the middle showed the naked eye observation results of the enlarged observation area, which showed the difference of skin wrinkles between week 0 and week 4 without using the VISIA high-end digital skin analyzer. The photos at the bottom showed the detection results of the photos in the middle of FIG. 7A, and the photos at the bottom were displayed by the VISIA high-end digital skin analyzer. In the photos at the bottom of FIG. 7A, as compared with week 0, the photo of facial skin at week 4 showed significantly fewer position marks of lengths and depths of wrinkles in the same area of the facial skin.

Referring to FIG. 7B, the mean skin wrinkles of the 10 subjects before dosing (week 0) were considered as 100%. After dosing with the red rice ferment every day for 4 consecutive weeks, the mean skin wrinkles of the 10 subjects were 83%. That is, dosing with 6.5 mL of red rice ferment every day effectively improved the skin wrinkles by up to 17%. Based on this, the red rice ferment had the effects of improving subject's skin wrinkles and resisting wrinkles. In FIG. 7B, * represents a significant difference (p<0.05) as compared with the skin wrinkles of the subjects at week 0.

(6-2-3) Skin Pore Detection

(a) Measurement Method

The facial skin of the subjects was detected using the VISIA high-end digital skin analyzer sold by Canfield, USA. The same facial skin of the same subject before and after the test was photographed under visible light (white light) using the high-resolution camera to obtain photos of the facial skin, and analysis was performed according to the number of large pores on the skin using built-in software of the skin analyzer to obtain the corresponding quantified skin pores. The higher the measured value, the greater the number of large pores.

(b) Experimental Results

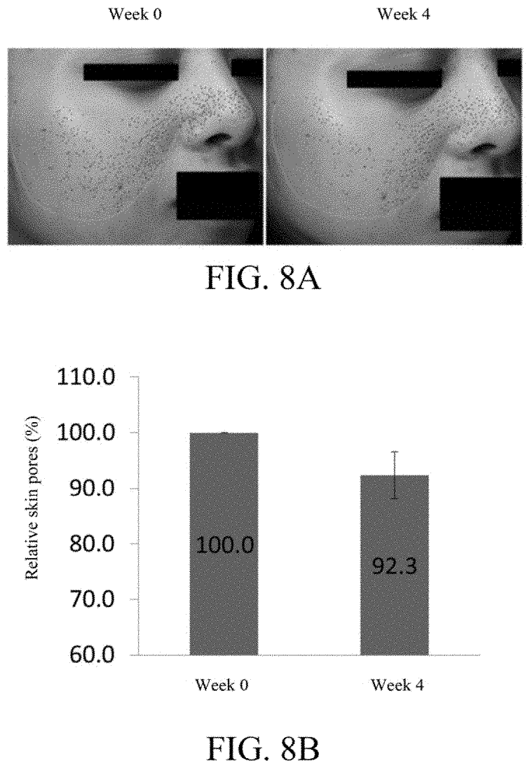

FIG. 8A showed photos of skin pores detection results of one of the subjects. As compared with week 0, the photo of facial skin at week 4 showed significantly fewer position marks of large pores detected in the same area of facial skin.

Referring to FIG. 8B, the mean skin pores of the 10 subjects before dosing (week 0) were considered as 100%. After dosing with the red rice ferment every day for 4 consecutive weeks, the mean skin pores of the 10 subjects were 92.3%. That is, dosing with 6.5 mL of red rice ferment every day effectively improved the skin pores by up to 7.7%. Based on this, the red rice ferment had the effects of alleviating subject's pores on skin and firming skin.

(6-2-4) Skin Redness Detection

(a) Measurement Method

The facial skin of the subjects was detected using the VISIA high-end digital skin analyzer sold by Canfield, USA. The facial skin was photographed using RBX polarized light technology of the skin analyzer to obtain photos of the facial skin, blood vessels or hemoglobin deep in the skin were detected, and measurement was performed according to the detection result using built-in software to obtain the skin redness. The higher the measurement value, the more severe the skin redness.

(b) Experimental Results

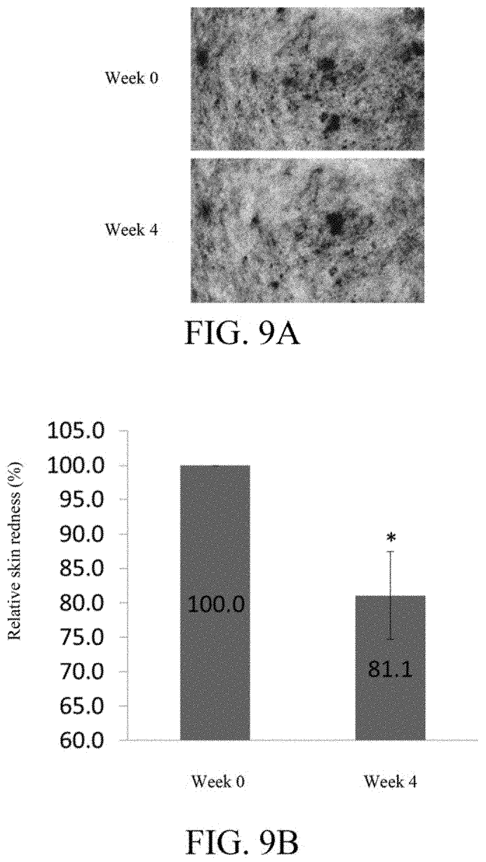

FIG. 9A showed photos of skin redness detection results of one of the subjects. As compared with week 0 (the darkness shown in the photo was the skin redness), the photo of facial skin at week 4 showed significantly less skin redness detected in the same area of facial skin.

Referring to FIG. 9B, the mean skin redness of the 10 subjects before dosing (week 0) was considered as 100%. After dosing with the red rice ferment every day for 4 consecutive weeks, the mean skin redness of the 10 subjects was 81.1%. That is, dosing with 6.5 mL of red rice ferment every day effectively improved the skin redness by up to 18.9%. Based on this, the red rice ferment had the effect of alleviating the skin redness of the subject. In FIG. 9B, * represents a significant difference (p<0.05) as compared with the skin redness of the subjects at week 0.

Based on the above, the preparation method of a red rice ferment according to any embodiment is suitable for preparing the red rice ferment with a skin care effect. In some embodiments, the total flavonoid content in the red rice ferment obtained after fermentation is greatly increased as compared with that before fermentation. In some embodiments, the red rice ferment also has the effects of scavenging free radicals, antioxidation and antiaging. In some embodiments, the red rice ferment can also promote skin cell proliferation and has the ability of accelerating skin tissue regeneration to strengthen skin barrier. In some embodiments, the red rice ferment can also enhance the ability of increasing the area of migration of human fibroblasts to the gap and has the effect of promoting repair function of human fibroblasts. In some embodiments, the red rice ferment can also enhance the mitochondrial activity of subject's skin cells, and thus have the antiaging effect of improving skin cells. In some embodiments, the red rice ferment can improve subject's skin texture. In some embodiments, the red rice ferment has the effects of alleviating one's skin wrinkles and preventing wrinkles. In some embodiments, the red rice ferment has the effects of alleviating subject's pores on skin and firming skin. In some embodiments, the red rice ferment has the effect of alleviating subject's skin redness.

Although the present invention has been described in considerable detail with reference to certain preferred embodiments thereof, the disclosure is not for limiting the scope of the invention. Persons having ordinary skill in the art may make various modifications and changes without departing from the scope and spirit of the invention. Therefore, the scope of the appended claims should not be limited to the description of the preferred embodiments described above.

What is claimed is:

1. A method of preparing a red rice ferment, comprising: fermenting a red rice extract by yeast, Lactobacillus, and Acetobacter aceti to obtain the red rice ferment.

2. The method of preparing a red rice ferment according to claim 1, wherein a ratio of fermentation time of the yeast to the Lactobacillus to the Acetobacter aceti is 1-2.5:1-3:3-10.

3. The method of preparing a red rice ferment according to claim 1, wherein the content of the yeast is 0.5% (w/v) of the red rice extract, the content of the Lactobacillus is 0.1% (w/v) of the red rice extract, and the content of the Acetobacter aceti is 5% (w/v) of the red rice extract.

4. The method of preparing a red rice ferment according to claim 1, further comprising extracting the red rice with water containing an enzyme solution to obtain the red rice extract.

5. The preparation method of a red rice ferment according to claim 4, wherein a volume ratio of the red rice to the water is 1:6, and the extraction is performed at a temperature of 60° C. to 100° C. for 0.5 hour to 5 hours.

6. A red rice ferment, obtained by the preparation method of a red rice ferment according to claim 1.

7. A method for improving skin condition of a subject in need thereof with the red rice ferment according to claim 6, comprising administering to the subject an effective dose of a composition comprising the red rice ferment.

8. The method according to claim 7, wherein the improvement of skin condition is selected from enhancing mitochondrial activity of skin fibroblasts, delaying skin aging, preventing wrinkles, alleviating skin redness, or a combination thereof.

9. The method according to claim 7, wherein the improvement of skin condition is selected from alleviating skin roughness, firming skin, minimizing skin pores, or a combination thereof.

10. The method according to claim 7, wherein the improvement of skin condition is selected from promoting regeneration of skin fibroblasts, promoting wound healing, reducing wound area, or a combination thereof.
